(19) United States
(12) Patent Application Publication
(10) Pub. No.: US 2024/0206955 A1
(43) Pub. Date: Jun. 27, 2024

(54) ELECTROSURGICAL PROBE AND ELECTRODE ASSEMBLY WITH AN ACTIVE ELECTRODE SUPPORT STRUCTURE

(71) Applicant: Arthrex, Inc., Naples, FL (US)

(72) Inventors: Jeffrey Haczynski, Naples, FL (US); Gabrielle Sereika, Bonita Springs, FL (US); Stephen Donnigan, Castle Rock, CO (US)

(73) Assignee: Arthrex, Inc, Naples, FL (US)

(21) Appl. No.: 18/528,996

(22) Filed: Dec. 5, 2023

Related U.S. Application Data
(60) Provisional application No. 63/434,684, filed on Dec. 22, 2022.

(51) Int. Cl. A61B 18/14

(52) U.S. Cl. CPC A61B 18/14; A61B 2018/00077

(57) ABSTRACT

An electrosurgical probe includes an elongated tubular shaft including a lumen extending along a body from a proximal shaft portion to a distal shaft portion. The distal shaft portion forms an active electrode as a first exposed conductive portion at a distal extent of the elongated shaft. A first insulating layer extends over the body from the proximal end portion to the distal end portion. A conductive layer extends over the first insulating layer and terminates proximal of the first insulating layer. A second insulating layer extends over the first insulating layer and the conductive layer and terminates proximal of a distal conducting portion of the conducting layer forming a return electrode.

### CROSS-REFERENCE TO RELATED APPLICATIONS

This application claims priority under 35 U.S.C. § 119(e) and the benefit of U.S. Provisional Application No. 63/434,684 entitled ELECTROSURGICAL PROBE AND ELECTRODE ASSEMBLY WITH AN ACTIVE ELECTRODE SUPPORT STRUCTURE, filed on Dec. 22, 2022, by Haczynski et al., the entire disclosure of which is incorporated herein by reference.

### BACKGROUND OF THE INVENTION

The present disclosure generally relates to an ablation apparatus or probe comprising one or more electrodes and a lumen for fluid transmission and, more particularly, to an electrode and probe assembly for an electrosurgical apparatus. In general, ablation apparatuses may be implemented in minimally invasive surgical operations. Such operations may limit trauma and tissue damage associated with various surgical procedures, thereby limiting patient recovery time and improving patient outcomes.

### SUMMARY

In general, the disclosure provides for ablation devices designed to deliver high-frequency signals to one or more electrodes to remove or manipulate tissue associated with a surgical procedure. Such devices may include various designs configured to access cavities or internal anatomical features of patients by being implemented as distally positioned accessories in connection with elongated probes. The disclosure generally provides for an assembly configuration and corresponding methods of manufacture for an ablation apparatus with improved operation and limited manufacturing variation. In various implementations, the disclosure provides for electrode assemblies for ablation devices that implement a variety of manufacturing methods and features to provide improved operation and longevity in operation. Though many of the exemplary devices disclosed include specific features in combination, it shall be understood that the features may be implemented in various combinations.

In various embodiments, the disclosure provides for an electrosurgical probe and electrode assembly comprising a tubular elongated shaft having a lumen extending along a longitudinal axis from a proximal shaft portion to a distal shaft portion. The distal shaft portion of the elongated shaft forms an active electrode as an exposed portion of a conductive exterior surface of the elongated shaft at a distal extent. A return electrode of the probe may be provided by a conductive layer that extends over a first insulating layer from the proximal shaft portion to the distal shaft portion. The first insulating layer may be interposed between the conductive layer and the conductive exterior surface of the elongated shaft, thereby insulating the conductive path associated with the elongated shaft and the active electrode from the conductive layer associated with the return electrode. The return electrode may be formed as an exposed conductive portion of the conductive layer which may extend distally beyond a second insulating layer. Additionally, the active electrode may be conductively and thermally insulated from the return electrode formed by the conductive layer via an insulator ring interposed therebetween. The insulator ring may extend over a distal insulating portion of the first insulating layer as well as a portion of the elongated shaft proximal of the active electrode.

These and other features, objects and advantages of the present disclosure will become apparent upon reading the following description thereof together with reference to the accompanying drawings.

### BRIEF DESCRIPTION OF THE DRAWINGS

FIG. 1 is a projected view of an ablation device demonstrating a control console and an electrosurgical probe;

FIG. 2 is a detailed projected view of an electrode assembly of an electrosurgical probe;

FIG. 3 is a projected view of an exemplary electrode assembly of an electrosurgical probe;

FIG. 4 is a cross-sectional view of the electrosurgical probe demonstrated in FIG. 3;

FIG. 5 is a partial cross-sectional view of an electrosurgical probe demonstrating a conductive connection of an electrode assembly;

FIG. 6 is a projected view of an exemplary electrode assembly of an electrosurgical probe;

FIG. 7 is a cross-sectional view of the electrosurgical probe demonstrated in FIG. 6;

FIG. 8 is a detailed cross-sectional view demonstrating a formed distal end portion of a supply electrode of an electrosurgical probe;

FIG. 9 is a partial cross-sectional view of an electrosurgical probe demonstrating a conductive connection of the electrode assembly;

FIG. 10 is a projected cross-sectional view of an electrosurgical probe demonstrating a user interface;

FIG. 11 is a side cross-sectional view of the user interface demonstrated in FIG. 10; and

FIG. 12 is an illustrative diagram of an electrosurgical ablation system and probe in accordance with the disclosure.

### DETAILED DESCRIPTION OF THE PREFERRED EMBODIMENT

In the following description of the preferred implementations, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

Ablation devices and electrosurgical systems may provide beneficial utilities for minimally invasive surgical procedures. Such procedures may limit patient recovery times and improve outcomes by applying specialized surgical techniques and tools to remotely access various treatment sites. As discussed in the following description, the disclosure provides for a variety of features for ablation devices, particularly in relation to electrode assemblies with improved manufacturability and related improvements in performance and reliability. In some examples, the electrode assemblies described herein may provide for layered structures that may be molded, formed, and/or printed successively over rigid conductive shaft structures to provide bipolar electrosurgical probes. In some cases, the conductive, rigid structure forming the electrosurgical probes may comprise an elongated, hollow conductive shaft that may provide for a conductive connection to a distal end portion of an electrosurgical probe as well as form an active or return electrode. The active or return electrode may correspond to an exposed, conductive exterior surface of the elongated shaft. As discussed further in the following examples, the successively layered structures or features forming the electrode assemblies may be implemented in various combinations with the elongated conductive shaft to suit a variety of applications.

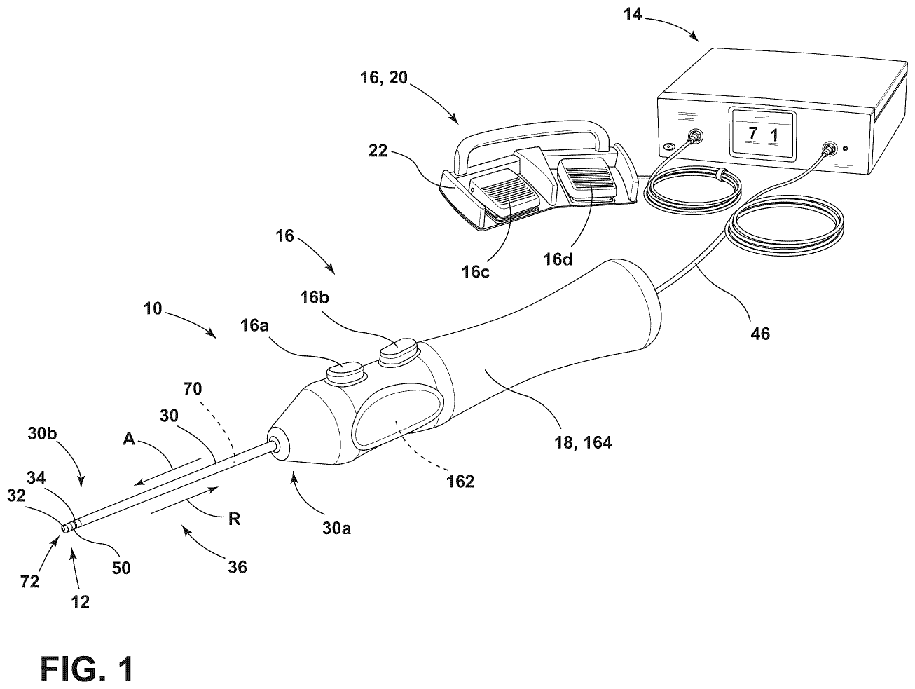

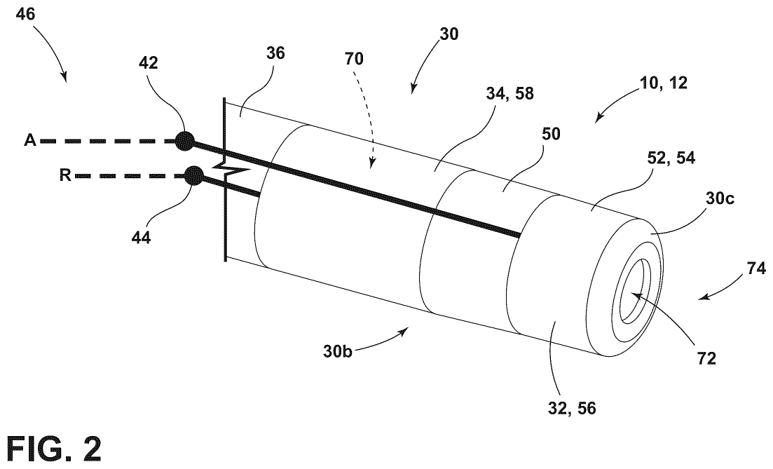

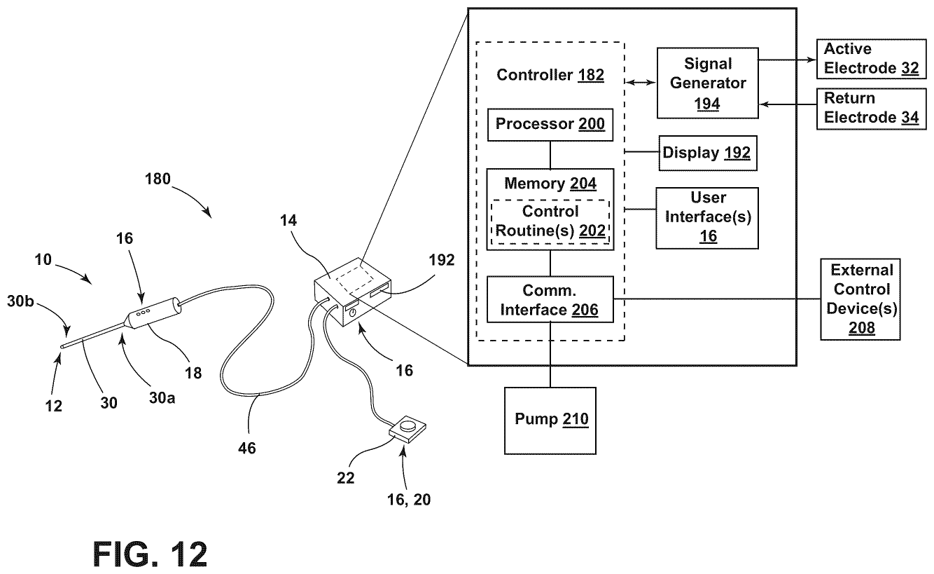

Referring now to FIGS. 1 and 2, an example of an ablation wand or electrosurgical probe 10 is shown demonstrating an electrode assembly 12 configured to deliver an ablation therapy based on control signals communicated by a control console 14. As generally demonstrated in FIG. 1, the probe 10 may comprise a user interface 16 that may be incorporated on a handle 18 of the probe 10 and/or separately connected to the control console 14 as a control accessory 20, exemplified by a foot pedal 22. As discussed in further detail in reference to FIG. 12, the settings of the control console 14 may be controlled in response to an actuation of one or more of a first input 16a, a second input 16b, a third input 16c, a fourth input 16d, etc. of the user interface 16. Various settings may be adjusted or controlled in response to an interaction with the user interface 16, some of which may include the adjustment of an intensity or frequency of an ablation control signal and/or the control of one or more devices in communication with the control console 14. In this configuration, the operation of the electrosurgical probe 10 may be updated during a surgical procedure to facilitate a variety of electrosurgical techniques.

As discussed in the following detailed examples, the electrode assembly 12 may be incorporated on an elongated shaft 30 in connection with the handle 18 and extend from a proximal shaft portion 30a to a distal shaft portion 30b. In various implementations, the electrode assembly 12 may correspond to a bipolar electrode assembly comprising a supply/active electrode 32 and a return/passive electrode 34 disposed on the distal shaft portion 30b. Accordingly, in order to effectuate an ablation therapy with the electrode assembly 12, control signals from the control console 14 may be communicated along a length or body 36 of the elongated shaft 30 to the electrode assembly 12 at the distal shaft portion 30b. As demonstrated symbolically in FIGS. 1 and 2, a transmission control signal A may be communicated to the supply electrode 32 through the elongated shaft 30 via a first conductive connection 42. A return signal R or feedback signal may be communicated from the return or passive electrode 34 through the elongated shaft via a second conductive connection 44. Each of the first and second conductive connections 42, 44 may conductively connect with the control console 14 via a flexible control cable 46. In various implementations, the disclosure provides for novel configurations of the conductive connections 42, 44 to provide for the communication of control signals A, R to and from the active and passive electrodes 32, 34 to provide ablation therapy to a surgical site. Such configurations may provide for improved performance of the electrosurgical probe 10 as well as improved manufacturing and quality control of the underlying electrode assemblies 12.

As demonstrated in FIG. 2, an insulator 50 may be interposed between the supply electrode 32 and the return electrode 34. In various implementations, the insulator 50 may be molded or formed over a portion of the elongated shaft 30 and may thermally and/or electrically insulate the supply electrode 32 from the return electrode 34. In some implementations, the supply electrode 32 or the return electrode 34 may be implemented on the electrode assembly 12 as an exposed portion 52 of an exterior surface 54 of the elongated shaft 30. In such implementations, the first or second conductive connections 42, 44 may be provided by conductively communicating the control signals A or R through the conductive material of the elongated shaft 30. For example, the second conductive connection 44 may be facilitated by the body 36 of the elongated shaft 30 through the conductive material (e.g., stainless steel) forming the elongated shaft 30. In other cases, the first conductive connection 42 may be provided by communication of transmission signals A through the body 36 of the elongated shaft 30. Accordingly, the disclosure provides for a variety of implementations of the electrode assembly 12 that may implement the conductive properties of the shaft 30 to provide for one of the conductive connections 42, 44.

In cases where the elongated shaft 30 provides the second conductive connection 44, the exposed portion 52 may correspond to the return electrode 34. As shown in FIG. 2, the first conductive connection 42 to the supply electrode 32 may also be facilitated by the exposed portion 52 of the elongated shaft 30. In various implementations, the elongated shaft 30 may provide for a conductive connection 42, 44 configured to communicate control signals A, R along a length of the body 36 of the elongated shaft 30. In operation, the control signals A, R may induce an ablation field proximate to the supply electrode 32 transmitted from a transmission surface 56 of the supply electrode 32 and received by the reception surface 58 of the return electrode 34. Accordingly, the disclosure may provide for the electrosurgical probe 10 to be implemented with at least a portion of the elongated shaft 30 forming the exposed portion 52 to implement the supply electrode 32 or the return electrode 34.

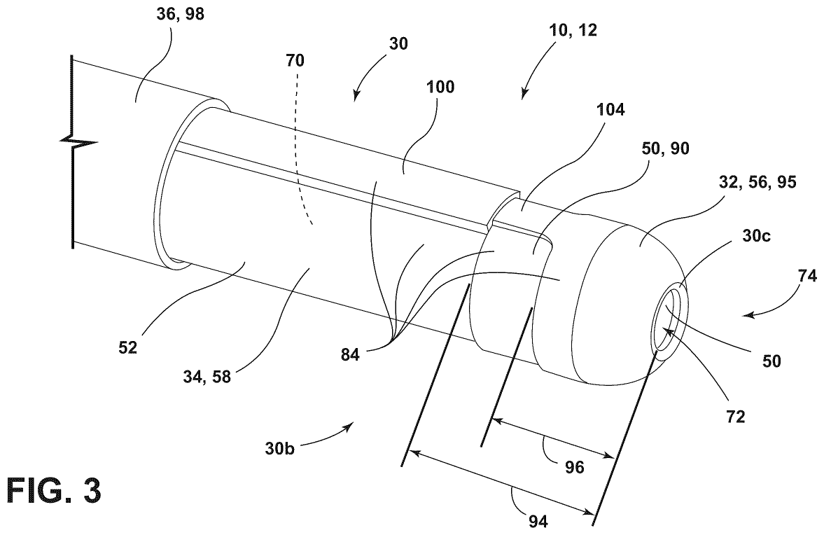

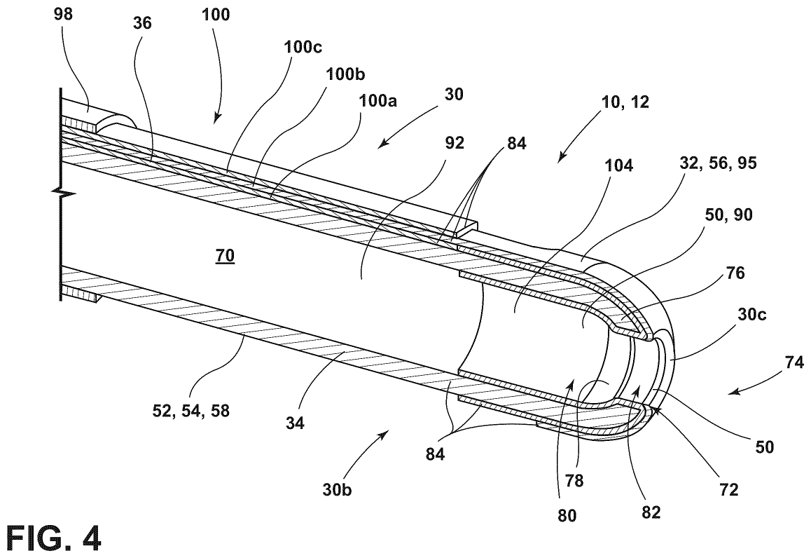

Referring now to FIGS. 3, 4, and 5, an implementation of the electrosurgical probe 10 is shown demonstrating the return electrode 34 formed by the exposed portion 52 corresponding to the reception surface 58 of the return electrode 34. As shown, an interior passage or lumen 70 is formed by the elongated shaft 30. At a distal extent 30c of the elongated shaft 30, an aperture or inlet orifice 72 may interconnect the lumen 70 with a treatment region 74 of the electrode assembly 12. In operation, the inlet orifice 72 may provide for aspiration or irrigation of the treatment region 74. In such implementations, the distal extent 30c of the elongated shaft 30 may have a torpedo-shaped distal end portion formed by radially converging walls 76 formed by a conductive tubular structure forming the elongated shaft 30. In this configuration, a distal end portion of the lumen 70 may form an interior dome-shaped wall 78 that tapers from a first diameter 80 of the lumen 70 to radially decrease to a second diameter 82 of the inlet orifice 72. The torpedo-shaped distal end portion may allow the distal extent 30c of the probe 10 to deliver ablation therapy to small spaces within a body cavity (e.g., a joint cavity).

As previously discussed, in some implementations, the supply electrode 32 may be formed via a plurality of alternating layers 84 of conductive material and insulating material formed on or disposed on the exterior surface 54 of the elongated shaft 30. In the example shown, a first insulating layer 90 may be formed over or coat the distal shaft portion 30b of the elongated shaft 30. The first insulating layer 90 may be formed over the exterior surface 54 as well as an interior surface 92 along the distal extent of the lumen 70 forming the inlet orifice 72. The first insulating layer 90 may extend a first length 94 along the exterior surface 54 and the interior surface 92 of the elongated shaft 30. In this configuration, the first insulating layer 90 may correspond to an insulating barrier extending over the distal extent 30c of the elongated shaft 30 that may be implemented to electrically insulate the supply electrode 32 from the conductive material of the elongated shaft 30.

As shown, the supply electrode 32 may be formed by a conductive layer 95 printed or applied over the first insulating layer 90. The conductive layer 95 may extend to a second length 96 from the distal extent 30c of the elongated shaft 30. The second length 96 of the supply electrode 32 may be less than the first length 94 of the first insulating layer 90. The difference between the first length 94 and the second length 96 may provide for the insulator 50 to extend between the supply electrode 32 applied over the first insulating layer 90 and the return electrode 34 formed by the exposed portion 52 of the elongated shaft 30. Accordingly, the exposed portion of the elongated shaft 30 may form the reception surface 58 of the return electrode 34. In this configuration, the return electrode 34 may extend proximal of the first length 94 of the first insulating layer 90.

Proximal of the return electrode 34, the body 36 of the elongated shaft 30 may be coated by a second insulating layer 98. The second insulating layer 98 may extend from the proximal shaft portion 30a to the distal shaft portion 30b. Additionally, an insulated, conductive connection may extend from the supply electrode 32 across the exposed portion 52 of the elongated shaft 30 formed by the reception surface 58 of the return electrode 34. The insulated conductive connection 100 may conductively connect to the supply electrode 32 and extend along the elongated shaft 30 from the distal shaft portion 30b to the proximal shaft portion 30a. As shown, the insulated conductive connection 100 may also be enclosed within the second insulating layer 98 proximal of the return electrode 34. In general, the insulated conductive connection 100 may provide for a conductive path for control signals to be communicated from the handle 18 and the control console 14 to the supply electrode 32. To complete the circuit from the supply electrode 32, the reception surface 58 of the return electrode 34 may provide a conductive path into the electrically conductive material of the elongated shaft 30 through which current may return to the handle 18 and control cable 46 of the control console 14.

The insulated conductive connection 100 may be formed by a plurality of alternating layers similar to the supply electrode 32 and the return electrode 34. As shown, the insulated conductive connection 100 may comprise an inner insulating layer 100a that may abut and extend from the first insulating layer 90 over the exposed portion 52 forming the return electrode 34. The conductive connection 100 may further comprise an encapsulated conductive layer 100b that may be printed or formed by conductive material in connection with the conductive layer 95 forming the supply electrode 32. An outer insulating layer 100c may be enclosed over the inner insulating layer 100a forming the insulated conductive connection 100 that extends along the body 36 of the elongated shaft 30. In this configuration, control signals communicated through the control cable 46 from the console 14 may be conducted from the handle 18 through the conductive connection 100 to supply activation energy to the supply electrode 32.

Each of the alternating layers 84 corresponding to the first insulating layer 90, the conductive layer 95, the second insulating layer 98, and the layers forming the conductive connection 100 may be sequentially applied to or formed over successive, exposed exterior surfaces of the elongated shaft 30. In some cases, the first insulating layer 90 may correspond to a dipped ceramic coating applied over the distal extent 30c of the elongated shaft. The inner insulating layer 100a of the insulated conductive connection 100 may extend over the exterior surface 54 along a concurrent layer of strata as the first insulating layer 90. The conducting layer 95 forming the supply electrode 32 may correspond to a second layer of strata extending over the first insulating layer 90 and the inner insulating layer 100a. In this configuration, the insulated conductive connection 100 may correspond to a printed conductive trace 104 conductively connecting the supply electrode 32 to an active connection terminal 106 at the proximal shaft portion 30a.

The outer insulating layer 100c may be printed or formed over the inner insulating layer 100a. The combination of the inner insulating layer 100a and the outer insulating layer 100c may complete the encapsulation of the conductive layer 95 extending therebetween. The insulating layers 100a, 100c may conductively insulate the conductive layer 95 of the supply electrode 32 from the conductive body 36 of the elongated shaft 30. Finally, proximal of the return electrode 34 formed by the exposed portion 52, the second insulating layer 98 may be formed as an encapsulated tube (e.g., heat shrink tube, polymeric coating, etc.) that may extend along a length of the body 36 from the proximal shaft portion 30a to the distal shaft portion 30b. In this configuration, the alternating layers 84 may provide for improved assembly and manufacture of the electrosurgical probe 10 while maintaining optimum performance.

As discussed herein, the manufacturing processes, materials, and resulting structures of the conductive layers, traces, and insulating layers may be implemented in a variety of ways. For example, a conductive layer (e.g., conductive layer 100b, conductive trace 104) may be formed by applying or extruding layers of conductive material in corresponding profile shapes and geometries with one or more compatible printing or modeling processes. In some instances, one or more infused polymer composites (e.g., polymer/carbon nanomaterial filaments) may be printed by an extrusion process (e.g., fused deposition modelling [FDM], direct ink writing [DIW], etc.). Accordingly, a printed structure may be prepared with one or more of several types of metallic composites, ceramic composites, carbon-based materials, such as graphite, graphene, nanotubes, or nanobuds, etc. Insulating layers (e.g., insulating layer 100c) may also be printed or applied to form the assemblies discussed herein using various processes, some of which may be implemented to provide alternating conducting and insulating layers. For example, a printed insulating layer may be prepared by applying one or more printed insulating materials (e.g., ceramic, polymers, etc.) in conjunction with the underlying surfaces and structures described herein. Printing processes may vary based on the specific application and may include various procedures including but not limited to lithography-based manufacturing, DIW, FDM, etc. Accordingly, the disclosure may provide for the electrosurgical probes 10 to be implemented in a variety of ways.

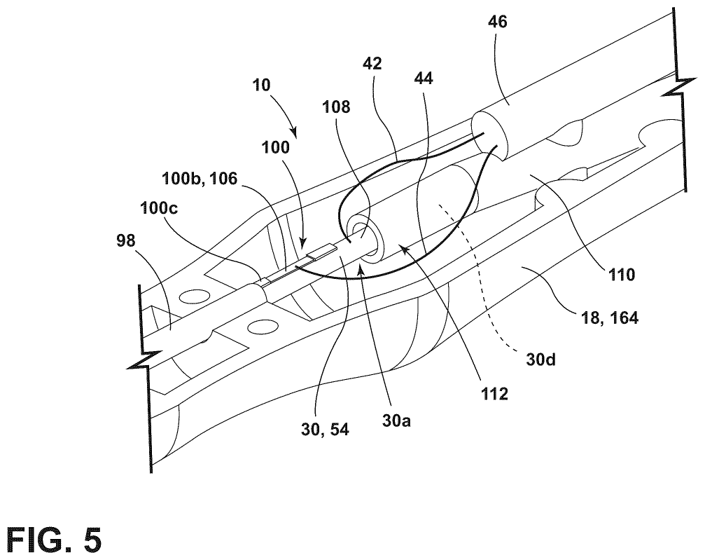

Referring now to FIG. 5, the active connection terminal 106 is demonstrated as a portion of the encapsulated conductive layer 100b exposed and in connection with the control cable 46. Additionally, a return connection terminal 108 may be provided by the control cable 46 conductively connecting to the exterior surface 54 of the proximal shaft portion 30a of the elongated shaft 30. Each of the active and return connection terminals 106, 108 may be incorporated within an insulating body or housing of the handle 18. Additionally, though hidden by the exterior surface 54, the lumen 70 may extend through the elongated shaft 30 to a proximal extent 30d. In this configuration, the proximal extent 30d of the elongated shaft 30 may interconnect the lumen 70 with a fluid transmission or suction conduit 110, which may be implemented to provide for irrigation and/or aspiration as discussed herein. Similar to the terminal connections 106, 108, an interface formed between the fluid transmission conduit 110 and the proximal extent 30d of the elongated shaft 30 may be enclosed within the housing formed by the handle 18.

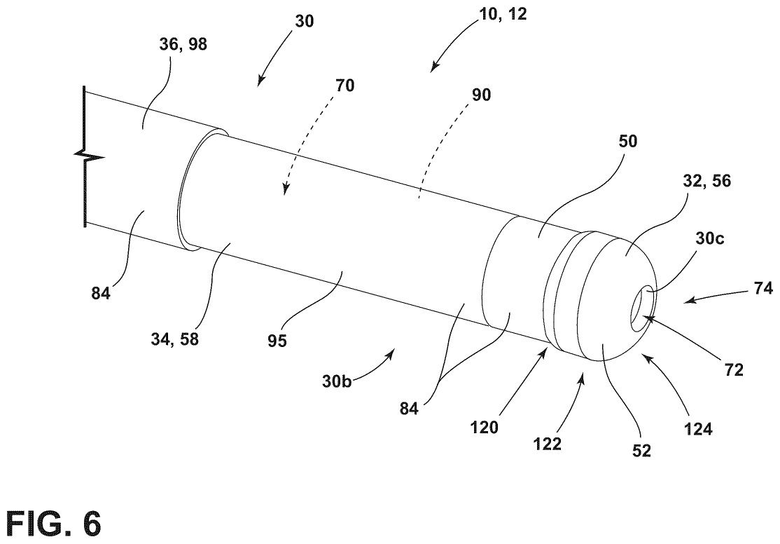

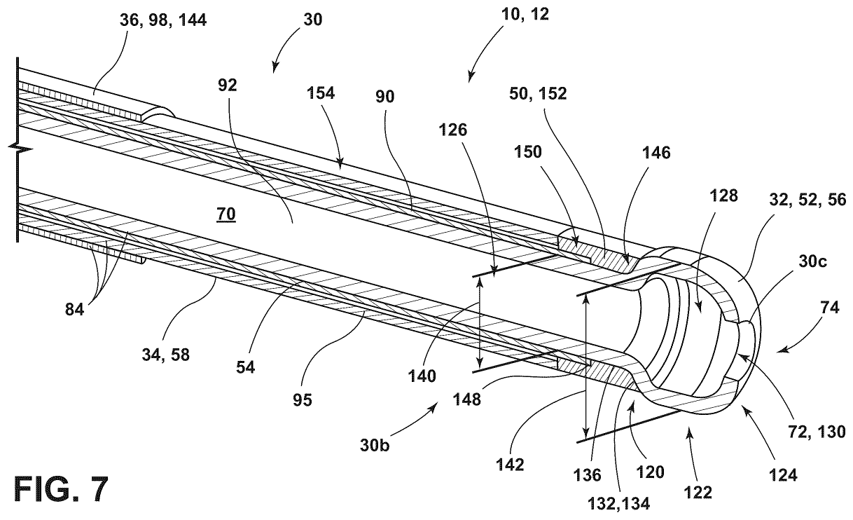

Referring now to FIGS. 6-9, an example of the electrosurgical probe 10 comprising the transmission surface 56 of the supply electrode 32 formed by the exposed portion 52 is shown. As previously discussed, like reference numerals may be applied to similar features throughout the disclosure. Accordingly, the alternating insulating or conducting layers 84 may similarly be implemented in cases where the conductive body 36 of the elongated shaft 30 is utilized to form the transmission surface 56 of the supply electrode 32. As best shown in FIGS. 6 and 7, the distal shaft portion 30b of the elongated shaft 30 may comprise an outward flare 120 forming a bulbous head 122 terminating at a rounded or dome-shaped end 124 at the distal extent 30c. As shown in FIG. 6, the outward flare 120 may increase the proportions of the bulbous head 122 and the lumen 70 proportionally at the inlet orifice 72 from a first diameter 126 or shaft diameter to a larger, second diameter 128 or head diameter. At the distal extent 30c, the lumen 70 may be fluidically connected to the treatment regions 74 via the inlet orifice 72, which may decrease to a third diameter 130 or orifice diameter. In this configuration, the lumen may expand radially outward and conform to an exterior profile shape 132 of the bulbous head 122 formed by the outward flare 120 and the dome-shaped end 124. As shown, the bulbous head 122 may form the transmission surface 56 of the supply electrode 32 as the exposed portion 52 of the elongated shaft 30.

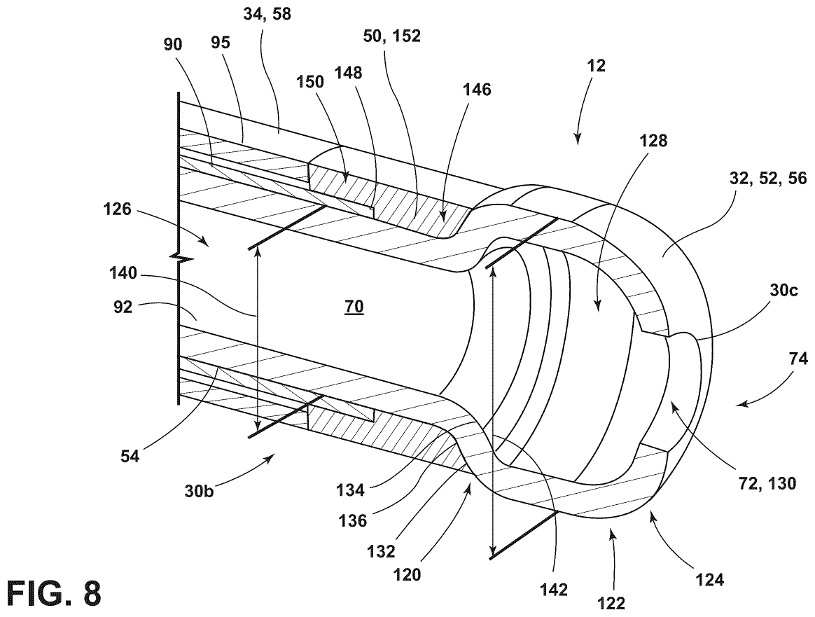

The outward flare 120 of the distal shaft portion 30b may be formed by deforming a tubular structure of the elongated shaft 30 via a die-forming operation applied to the distal shaft portion 30b. Alternatively, the outward flare 120 and bulbous head 122 may be formed via a variety of machining, metal forming, welding, or various other fabrication operations. As demonstrated in FIGS. 7 and 8, the outward flare 120 may form a proximal end portion of the bulbous head 122 via a gradual transition profile 134 that may correspond to a serpentine or sinusoidal transition between the body 36 of the shaft 30 and the transmission surface 56 of the bulbous head 122 forming the supply electrode 32. Proximal of the bulbous head 122, a recess 136 or circumferential recess may be formed about a cylindrical perimeter of the transition profile 34 radially about the exterior surface 54 of the shaft 30. As provided in the following exemplary description, the alternating layers 84 of the electrosurgical probe 10 may extend along the body 36 of the shaft 30 within the recess 136 or draft of the bulbous head 122. As shown, the alternating insulating and conducting layers 84 may be disposed on or formed over a first outside diameter 140 of the exterior surface 54 along the body 36 and the recess 136 a draft formed by a second outside diameter 142 of the bulbous head 122. In this configuration, the alternating layers 84 forming the return electrode 34 and an insulated exterior body 144 of the shaft 30 may be formed within a radial gap 146 between the first outside diameter 140 and the second outside diameter 142. Accordingly, a clearance profile or clearance passage through which the electrosurgical probe 10 may be inserted may be defined by the second outside diameter 142 of the bulbous head 122 because the proximal or trailing features of the electrode assembly 12 may have a diameter less than or equal to the second outside diameter 142 of the bulbous head 122.

Referring still to FIGS. 7 and 8, the first insulating layer 90 may extend from the proximal shaft portion 30a of the elongated shaft 30 to the distal shaft portion 30b proximal of the outward flare 120. The conducting layer 95 may similarly extend from the proximal shaft portion 30a and may terminate proximal of a distal extent 148 of the first insulating layer 90. In this configuration, the distal extent 148 may provide for an insulating overlap region 150 between the first insulating layer 90 and an insulator ring or sleeve 152 forming the insulator 50 between the supply electrode 32 and the return electrode 34. The insulating sleeve 152 may thermally and conductively insulate the transmission surface 56 of the supply electrode 32 from the reception surface 58 of the return electrode 34 formed by a distal exposed portion 154 of the conducting layer 95. Additionally, the insulating sleeve may serve as a thermally insulating barrier between the supply electrode 32 and the material forming the first insulating layer 90, which may correspond to a polymeric material with limited heat resistance. In this configuration, the insulator ring 152 may be formed over the insulating overlap region 150 and abut and interpose the distal exposed portion 154 forming the return electrode 34 and the outward flare 120 and bulbous head 122 forming the supply electrode 32.

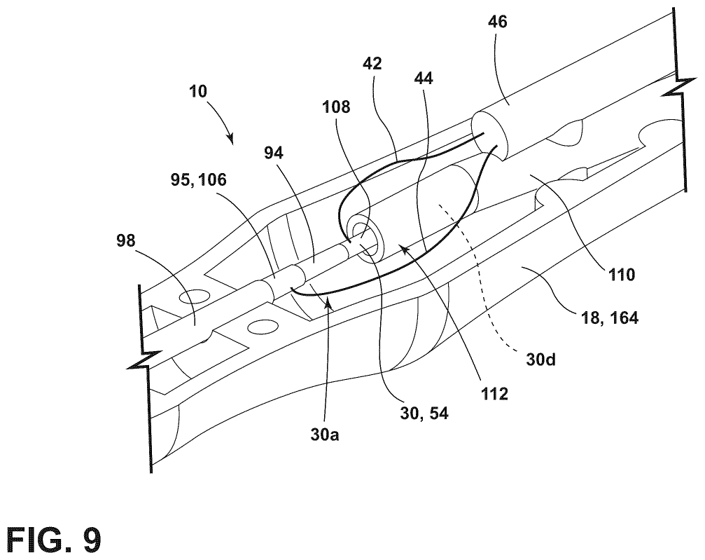

While the insulator ring 152 may abut a distal end of the return electrode 34, a proximal extent of the return electrode 34 may be defined by a distal extent of the second insulating layer 98. As shown, the reception surface 58 of the return electrode 34 extends between the distal extent of the second insulating layer 98 and the proximal end of the insulator ring 152. The second insulating layer 98 may be implemented by various electrically insulating materials including various ceramics or polymers that may be applied as coatings, wraps, nested structures, etc. In some implementations, the second insulating layer 98 may comprise a heat-molded polymeric material or thermoplastic material such as polyolefin, fluoropolymers (e.g., FEP, PTFE or Kynar), PVC, neoprene, silicone elastomer, Viton, etc. Though not shown in FIG. 6, the conducting layer 95 and the second insulating layer 98 may extend along the body 36 of the shaft 30 to the proximal shaft portion 30a demonstrated in FIG. 9.

Referring now to FIG. 9, the active connection terminal 106 and the return connection terminal 108 are demonstrated in conductive connection with the exterior surface 54 of the elongated shaft 30 and the conducting layer 95, respectively. As previously discussed, the control cable 46 in communication with the control console 14 may communicate control signals to the active connection terminal 106 and monitor return signals communicated via the return connection terminal 108 throughout operation of the electrosurgical probe 10. As discussed previously, the proximal extent 30d of the elongated shaft 30 is shown in connection with the fluid transmission conduit 110. In this configuration, irrigation or aspiration of fluids communicated through the lumen 70 may be transmitted through the fluid transmission conduit 110 to facilitate operation of the electrosurgical probe 10. Each of the connection terminals 106, 108 and an interface 112 between the elongated shaft 30 and the fluid transmission conduit 110 may be housed within the housing formed by the handle 18.

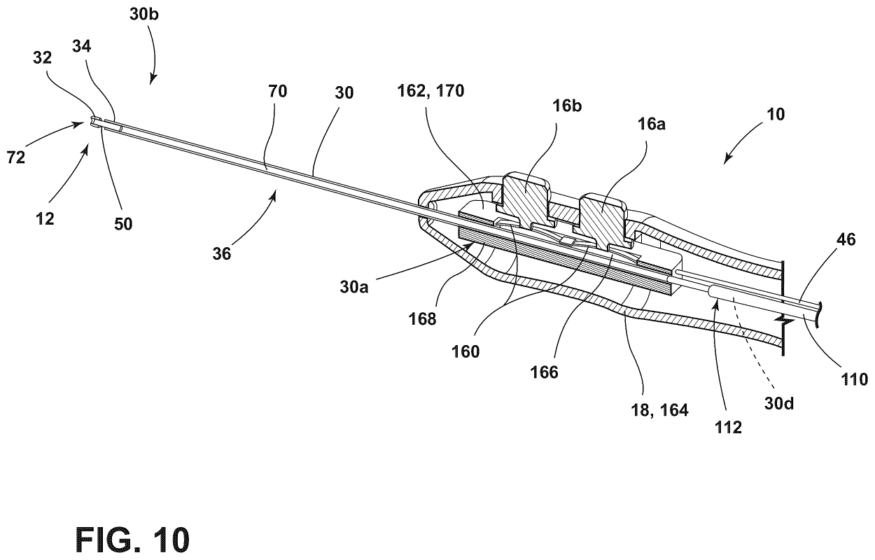

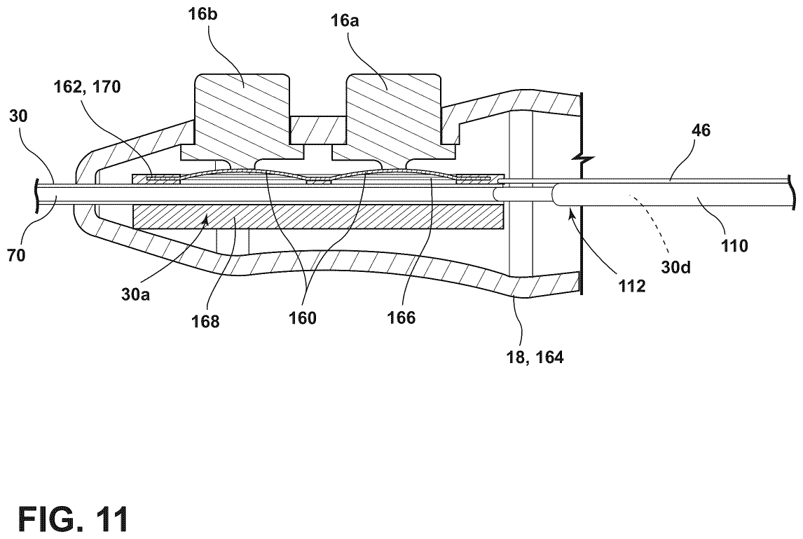

Referring now to FIGS. 10 and 11, cross-sectional views of the handle 18 and the proximal shaft portion 30a of the electrosurgical probe 10 are shown demonstrating exemplary first and second inputs 16a, 16b of the user interface 16. In some implementations, the inputs 16a, 16b forming the user interface 16 may be implemented as spring switches 160 that may be anchored to a handpiece control module 162 in a housing 164 of the handle 18. In various implementations, the handpiece control module 162 may comprise a control circuit 166 that may be enclosed or encapsulated within a barrier layer 168, which may correspond to a potted or molded polymeric barrier. The control circuit 166 may further be connected to the proximal shaft portion 30a. In this configuration, the interface of the spring switches 160 with the control circuit 166 may be connected to the elongated shaft 30 and encapsulated within the barrier layer 168 to prevent contamination or water ingress related to the operating environment of the electrosurgical probe 10. The active and return connection terminals 106, 108 may also be encapsulated within the barrier layer 168, such that the control cable 46 may be connected to an encapsulated assembly 170 formed by the control circuit 166 and the proximal shaft portion 30a, each enclosed within the barrier layer 168. Proximal of the encapsulated assembly 170 and within the housing 164 formed by the handle 18, the conduit interface 112 may extend from the proximal extent 30d of the elongated shaft 30 for connection to the fluid transmission conduit 110. In this configuration, the control cable 46 and the fluid transmission conduit 110 may interconnect the conduit interface 112 and the connection terminals 106, 108 to conductively connect the control circuit 166 and the electrodes 32, 34 of the electrosurgical probe 10 with the control console 14 and an irrigation or aspiration pump associated with the lumen 70.

In various implementations, the spring switches 160 may correspond to cantilever beam switches that provide for normally open contacts with the control circuit 166. The springs of the cantilever beams or spring switches 160 may provide for the corresponding buttons of the user interface 16 (e.g., first input 16a, second input 16b) to have a compressive travel distance associated with the deflection of the spring that may provide tactile feedback upon depression by a user. In this configuration, the spring switches 160 may be formed in connection with the encapsulated assembly 170 preventing malfunction that may result from fluid or particulate contamination. As shown, the buttons forming the inputs 16a, 16b may extend through button apertures 172 formed through the housing 164 of the handle 18. In this way, the inputs 16a, 16b may provide for interaction with the handpiece control module 162 while preventing damage or malfunction of the control module 162 and the underlying circuitry or conductive connections.

Referring now to FIG. 12, a block diagram of an electrosurgical system 180 or ablation system is shown demonstrating the electrosurgical probe 10 and the control console 14. As previously discussed, the electrosurgical probe 10 may be in communication with the control console 14 via the control cable 46. The control console 14 or module may comprise a controller 182 that may control the delivery of signals to the supply electrode 32 and/or control a fluid flow rate (e.g., an aspiration rate) through the lumen 70. In operation, the controller 182 may receive inputs via a user interface 16, which may be distributed among a control console 14 as well as one or more external control accessories 20. The external control accessories 20 may correspond to one or more electronic or electromechanical buttons, triggers, foot pedals 22, or additional peripherals and devices communicatively connected to the control console 14. The user interface 16 may include one or more switches 16a, 16b, buttons, dials, and/or displays, which may include soft-key or touchscreen devices incorporated in a display device 192 (e.g., liquid crystal display [LCD], light emitting diode [LED] display, cathode ray tube [CRT], etc.). In response to inputs received from the user interface 16, the controller 182 may activate or adjusts the settings of the control signals communicated to the electrosurgical probe 10. The control signals generated by control console 14 may be controlled by a signal generator 194 configured to generate periodic or RF signals that induce an ablation treatment field (e.g., an electric or RF field) in response to control instructions (e.g., timing signals, amplification, etc.) communicated from the controller 182. The control signals may be communicated from the signal generator 194 of the control console 14 to the electrosurgical probe 10 via the control cable 46.

The control cable 46 may be conductively connected to the active or supply electrode 32 to transmit the output control signal A and connected to the return electrode 34 to receive a return signal R. The return signal R may be monitored by the controller 182 to provide closed-loop feedback to adjust the control signal A. The control signal A from the signal generator 194 may correspond to an AC driving signal generated in response to time-modulated signals from a processor 200 or clock of the controller 182. The AC driving signal may induce the treatment or ablation field in the form of RF energy. The modes of operation of the electrosurgical probe 10 may be controlled by adjusting the amplitude of the voltage and timing of the signal modulation that instructs the signal generator 194 to generate RF signals. Accordingly, by adjusting the voltage potential and the frequency or timing characteristics of the AC driving signal output from the signal generator 194, the controller 182 may control the operation of the electrosurgical probe 10 in response to inputs received via the user interface 16. In some embodiments, the controller 182 may be configured to activate one or more preset modes (e.g. ablation, coagulation) and the associated power levels or frequencies as presets in response to inputs received from the user interface 16.

The processor 200 of the controller 182 may be implemented as a microprocessor, microcontroller, application-specific integrated circuit (ASIC), or other circuitry configured to perform instructions, computations, and control various input/output signals to control the system 180. The instructions and/or control routines 202 of the system 180 may be accessed by the processor 200 via a memory 204. The memory 204 may comprise random access memory (RAM), read only memory (ROM), flash memory, hard disk storage, solid state drive memory, etc. The controller 182 may incorporate additional communication circuits or input/output circuitry. In an exemplary embodiment, a communication interface 206 of the controller 182, may include digital-to-analog converters, analog-to-digital converters, digital inputs and outputs, as well as one or more peripheral communication interfaces or buses. The peripheral communication interfaces of the communication interface 206 may be implemented with various communication protocols, such as serial communication (e.g., CAN bus, I2C, etc.), parallel communication, network communication (e.g., RS232, RS485, Ethernet), wireless network communication (Wi-Fi, 802.11, etc.). In some examples, the controller 182 may be in communication with one or more external devices 208 (e.g., control devices, peripherals, servers, etc.) via the communication interface 206. Accordingly, the control console 14 may provide for communication with various devices to update, maintain, and control the operation of the system 180.

Though not pictorially illustrated in the figures, a pump 210 (e.g., irrigation and/or aspiration pump) may be connected via one or more fluid conduits in connection with the lumen 70. In this way, the probe 10 may be configured to effectuate fluid transfer to/from the surgical site via the fluid transmission conduit 110. The pump 210 may be controlled via the user interface 16 of the controller 182 to adjust a flow rate, pressure, or intensity of the fluid transfer. The pump 210 may be implemented with a variety of pumping technologies (e.g., peristaltic, reciprocating, etc.) and may vary in fluid transfer capacity based on the application of the electrosurgical probe 10.

In some aspects, the disclosure may provide for an electrosurgical probe comprising an elongated shaft comprising a lumen extending along a longitudinal axis from a proximal shaft portion to a distal shaft portion. The distal shaft portion forms an active electrode as an exposed portion of the elongated shaft at a distal extent. An insulating sleeve extends around the distal shaft portion proximal of the exposed portion. An insulating layer is disposed on an exterior surface of the elongated shaft and extends from the proximal shaft portion to the insulating sleeve. A conductive layer is disposed over the distal shaft portion of the insulating layer. The conductive layer forms a return electrode insulated from the active electrode of the elongated shaft by the insulating sleeve.

According to various further aspects, the disclosure may implement one or more of the following features or configurations in various combinations:
the conductive layer is further insulated from the elongated shaft via the insulating layer;
the insulating layer is a first insulating layer of a plurality of insulating layers, the plurality of insulating layers further comprise a second insulating layer disposed over a proximal conductive portion of the conductive layer;
the second insulating layer extends from the proximal conductive portion of the return electrode to the proximal shaft portion of the elongated shaft;
the second insulating layer distally terminates exposing the return electrode formed by the conductive layer;
the elongated shaft forms a bulbous portion distal of the insulating sleeve;
the bulbous portion comprises a flared head having a second diameter that is larger than a first diameter of a body of the elongated shaft;
the insulating sleeve comprises a molded ring in connection with the insulating layer and interposed between the return electrode and the flared head of the elongated shaft;
the molded ring is formed of a thermally insulating material that limits heat transmission from the active electrode to the insulating layer;
the bulbous portion forms a dome-shaped end or rounded end that encloses radially about an orifice opening to the lumen at the distal extent of the elongated shaft;
a body of the elongated shaft extends along a first diameter and flares outward over a transition profile to a second diameter forming the bulbous head; and/or
the transition profile extends outward from the first diameter to the second diameter over a serpentine or sinusoidal transition.

According to another aspect, the disclosure may provide for a method for providing an electrosurgical probe. The method may include providing an elongated tubular shaft of an electrically conductive material having a body extending from proximal shaft portion to a distal shaft portion and a distal end having a bulbous portion flaring outward from the body forming an active electrode. A first insulating layer may be formed along a length of the body of the elongated tubular shaft. A conductive layer may be formed over the first insulating layer. A second insulating layer may extend from the proximal shaft portion to the distal shaft portion. The second insulating layer may terminate proximal of an exposed portion of the conductive layer, wherein the exposed portion of the conductive layer forms a return electrode.

According to various further aspects, the disclosure may implement one or more of the following features or configurations in various combinations:
the forming of the first insulating layer comprises forming the first insulating layer extending distally of the conductive layer as a distal insulating portion;
applying an insulating sleeve over the distal insulating portion and an exterior surface of the distal shaft portion proximal of the active electrode;
applying the insulating sleeve comprises molding a thermally and conductively insulating material over the distal insulating portion and the exterior surface of the distal shaft portion;
forming the first insulating layer and the second insulating layer comprises sequentially heat molding a first polymeric tube and a second polymeric tube over the elongated tubular shaft; and/or
the conducting layer is formed over the first polymeric tube and extends from the proximal shaft portion to the distal shaft portion.

According to yet another aspect, the disclosure may provide for an electrosurgical probe comprising an elongated tubular shaft comprising a lumen extending along a body from a proximal shaft portion to a distal shaft portion. The distal shaft portion forms an active electrode as a first exposed conductive portion at a distal extent of the elongated shaft. A first insulating layer extends over the body from the proximal end portion to the distal end portion. A conductive layer extends over the first insulating layer and terminating proximal of the first insulating layer, and a second insulating layer extends over the first insulating layer and the conductive layer, wherein the second insulating layer terminates proximal a second exposed conductive portion of the conducting layer. The second exposed conductive portion forms a return electrode. In some implementations, the electrosurgical probe may include an insulator ring formed over the distal end portion of the elongated tubular shaft and interposed between the return electrode and the active electrode. The insulator ring may extend over the distal insulating portion and the elongated tubular shaft proximal of the active electrode.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

1. An electrosurgical probe comprising:
an elongated shaft comprising a lumen extending along a longitudinal axis from a proximal shaft portion to a distal shaft portion, the distal shaft portion forming an active electrode as an exposed portion of the elongated shaft at a distal extent;
an insulating sleeve extending around the distal shaft portion proximal of the exposed portion;
an insulating layer disposed on an exterior surface of the elongated shaft and extending from the proximal shaft portion to the insulating sleeve; and
a conductive layer disposed over the distal shaft portion of the insulating layer, wherein the conductive layer forms a return electrode insulated from the active electrode of the elongated shaft by the insulating sleeve.

2. The electrosurgical probe according to claim 1, wherein the conductive layer is further insulated from the elongated shaft via the insulating layer.

3. The electrosurgical probe according to claim 1, wherein the insulating layer is a first insulating layer of a plurality of insulating layers, the plurality of insulating layers further comprising:
a second insulating layer disposed over a proximal conductive portion of the conductive layer.

4. The electrosurgical probe according to claim 3, wherein the second insulating layer extends from the proximal conductive portion of the return electrode to the proximal shaft portion of the elongated shaft.

5. The electrosurgical probe according to claim 3, wherein the second insulating layer distally terminates exposing the return electrode formed by the conductive layer.

6. The electrosurgical probe according to claim 1, wherein the elongated shaft forms a bulbous portion distal of the insulating sleeve.

7. The electrosurgical probe according to claim 6, wherein the bulbous portion comprises a flared head having a second diameter that is larger than a first diameter of a body of the elongated shaft.

8. The electrosurgical probe according to claim 7, wherein the insulating sleeve comprises a molded ring in connection with the insulating layer and interposed between the return electrode and the flared head of the elongated shaft.

9. The electrosurgical probe according to claim 8, wherein the molded ring is formed of a thermally insulating material that limits heat transmission from the active electrode to the insulating layer.

10. The electrosurgical probe according to claim 6, wherein the bulbous portion forms a dome-shaped end or rounded end that encloses radially about an orifice opening to the lumen at the distal extent of the elongated shaft.

11. The electrosurgical probe according to claim 6, wherein a body of the elongated shaft extends along a first diameter and flares outward over a transition profile to a second diameter forming the bulbous head.

12. The electrosurgical probe according to claim 11, wherein the transition profile extends outward from the first diameter to the second diameter over a serpentine or sinusoidal transition.

13. A method for providing an electrosurgical probe, the method comprising:
providing an elongated tubular shaft of an electrically conductive material having a body extending from proximal shaft portion to a distal shaft portion and a distal end having a bulbous portion flaring outward from the body forming an active electrode;
forming a first insulating layer along a length of the body of the elongated tubular shaft;
forming a conductive layer over the first insulating layer;
forming a second insulating layer extending from the proximal shaft portion to the distal shaft portion; and
terminating the second insulating layer proximal of an exposed portion of the conductive layer, wherein the exposed portion of the conductive layer forms a return electrode.

14. The method according to claim 13, wherein the forming of the first insulating layer comprises forming the first insulating layer extending distally of the conductive layer as a distal insulating portion.

15. The method according to claim 13, further comprising:
applying an insulating sleeve over the distal insulating portion and an exterior surface of the distal shaft portion proximal of the active electrode.

16. The method according to claim 15, wherein applying the insulating sleeve comprises molding a thermally and conductively insulating material over the distal insulating portion and the exterior surface of the distal shaft portion.

17. The method according to claim 16, wherein forming the first insulating layer and the second insulating layer comprises sequentially heat molding a first polymeric tube and a second polymeric tube over the elongated tubular shaft.

18. The method according to claim 13, wherein the conducting layer is formed over the first polymeric tube and extends from the proximal shaft portion to the distal shaft portion.

19. An electrosurgical probe comprising:
an elongated tubular shaft comprising a lumen extending along a body from a proximal shaft portion to a distal shaft portion, the distal shaft portion forming an active electrode as a first exposed conductive portion at a distal extent of the elongated shaft;
a first insulating layer extending over the body from the proximal end portion to the distal end portion;
a conductive layer extending over the first insulating layer and terminating proximal of the first insulating layer; and
a second insulating layer extending over the first insulating layer and the conductive layer, wherein the second insulating layer terminates proximal a second exposed conductive portion of the conducting layer, wherein the second exposed conductive portion forms a return electrode.

20. The electrosurgical probe according to claim 19, further comprising:
an insulator ring formed over the distal end portion of the elongated tubular shaft and interposed between the return electrode and the active electrode, wherein the insulator ring extends over the distal insulating portion and the elongated tubular shaft proximal of the active electrode.
